(19) United States
(12) Patent Application Publication
(10) Pub. No.: US 2025/0195585 A1
(43) Pub. Date: Jun. 19, 2025

(54) POSTBIOTIC COMPOSITIONS AND METHODS AND USE THEROF

(71) Applicant: Schism Bioworks LLC, Evergreen, CO (US)

(72) Inventors: Sebastian Balcombe, Broomfield, CO (US); Ralf Jäger, Whitefish Bay, WI (US); Martin Purpura, Spring, TX (US)

(73) Assignee: Schism Bioworks LLC, Evergreen, CO (US)

(21) Appl. No.: 18/986,463

(22) Filed: Dec. 18, 2024

Related U.S. Application Data
(60) Provisional application No. 63/611,383, filed on Dec. 18, 2023.

(51) Int. Cl. A61K 35/74; A61K 35/741; A61P 29/00

(52) U.S. Cl. CPC A61K 35/74; A61K 35/741; A61P 29/00

(57) ABSTRACT

Disclosed herein are number embodiments relating to technologies for the use of postbiotic materials for the improvement of health. In certain embodiments, the composition is a postbiotic derived from at least one strain of bacteria wherein the at least one strain of bacteria comprises Propionibacterium freudenreichii UF (UF1). In certain specific embodiments contained in this disclosure, the postbiotic materials can lead to an reduction in body weight and an increase the portion of weight lost from fat rather than lean body mass.

### CROSS-REFERENCE TO RELATED APPLICATION(S)

This application claims the benefit under 35 U.S.C. § 119 (e) to U.S. Provisional Application No. 63/611,383, filed Dec. 18, 2023, and entitled “POSTBIOTIC COMPOSITIONS AND METHODS AND USE THEREOF”, which is hereby incorporated herein by reference in its entirety.

### BACKGROUND

Obesity is a significant problem in the Western world, with estimates of its prevalence ranging from 30% to 50% of the middle-aged population. The number of overweight (defined as a person with a body mass index (BMI) equal to or greater than 25 kg/m²) and obese (defined as a person with a BMI equal to or greater than 30 kg/m²) Americans has continued to increase since 1960, a trend that is not slowing down. Today, approximately 64.5% of adult Americans (about 199 million) are categorized as being overweight or obese. Obesity is becoming a growing concern as the number of people with obesity continues to increase and more is learned about the negative health effects of obesity. Obesity can lead to type 2 diabetes, heart disease, and some cancers. Each year, obesity causes at least 300,000 deaths in the U.S., and healthcare costs for American adults with obesity amount to more than $147 billion. There is a need in the art for compositions and methods to more effectively achieve weight loss.

### BRIEF SUMMARY

Described herein are compositions for reducing body weight, reducing body fat, and/or preventing obesity and related conditions, and for conferring other health benefits, and/or treating other condition. In certain aspects, the disclosed methods for reducing body weight, reducing body fat, and/or preventing obesity and related conditions involve administration to subject in need thereof a composition comprising: a probiotic derived from at least one strain of bacteria wherein the at least one strain of bacteria comprises Propionibacterium freudenreichii UF (UF1).

Further disclosed herein are compositions for the treatment of conditions at least one of obesity, immune system function associated disorders, insulin secretion associated disorders, diabetes, virulence associated conditions, cardiovascular disorder, cardiac disorders, degenerative diseases, sarcopenia, ocular disease, fibrotic diseases, aging associated disorders, improving skin hydration and collagen synthesis, liver disease, and Crohn's disease.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE FIGURES

FIG. 1 is a graph showing the results of Example 1, according to one implementation.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

As used herein, the term “postbiotic” refers to non-viable bacterial products (either intact cells or cell fragments) or metabolic products from microorganisms that have biological activity when administered in adequate amounts to a subject.

As used herein, the term “probiotic” can refer to a composition containing at least one species, genus, family, strain, order, or class of probiotic bacteria (e.g., a single isolate or a combination of desired bacteria), and can also include any additional carriers, excipients, and/or therapeutic agents that can be administered to a mammal. In certain embodiments, the probiotic composition comprises a buffering agent to allow the probiotic bacteria to survive in the acidic environment of the stomach, that is, the probiotic bacteria resist low pH and are able to survive passage through the stomach to colonize and grow in the intestinal milieu. Buffering agents can include, for example, sodium bicarbonate, milk, yoghurt, infant formula, and other dairy products. In certain embodiments, the probiotic composition is formulated as a food additive. In certain embodiments, the probiotic composition includes other materials known in the art for inclusion in food additives, such as water or other aqueous solutions, starch, binders, thickeners, colorants, flavorants, odorants, acidulants (e.g., lactic acid or malic acid, among others), vitamins, or minerals, among others.

As used herein, the term “subject” refers to the target of administration. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment of one or more body weight disorders prior to the administering step. In some aspects of the disclosed method, the subject has been diagnosed with a need for increasing weight loss prior to the administering step.

The term “overweight” is defined as the condition wherein the individual has a BMI greater than or 25 kg/m² and less than 30 kg/m². The terms “overweight” and “pre-obese” are used interchangeably.

As used herein, the term “obesity” is defined as the condition wherein the individual has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the term “class III obesity” is the condition wherein the BMI is equal to or greater than 40 kg/m².

As used herein, the terms “manage,” “managing,” and “management” encompass preventing, delaying, or reducing the severity of a recurrence of an adipose associated body composition or body weight disorder, such as obesity, lipodystrophy, diabetes or metabolic syndrome, fibrosis and cancer in a patient who has already suffered from such a disease, disorder or condition. The terms encompass modulating the threshold, development, and/or duration of the adipose associated body composition or body weight disorder, such as obesity, lipodystrophy, diabetes or metabolic syndrome, fibrosis and cancer or changing how a patient responds to the adipose associated body composition or body weight disorder.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with obesity” means having been subjected to a physical examination by a person of skill, for example, a physician, and found to have a condition that can be diagnosed or treated by a compound or composition that can reduce body mass. As a further example, “diagnosed with a need for weight loss” refers to having been subjected to a physical examination by a person of skill, for example, a physician, and found to have a condition characterized by excess of body fat or other disease wherein decreasing body fat would be beneficial to the subject. Such a diagnosis can be in reference to a disorder, such as obesity, metabolic syndrome, and the like, as discussed herein.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to excess adipose tissue) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein “inflammatory disease or condition” includes, but is not limited to: which the compositions and methods herein may be used include, but are not limited to: acquired immune deficiency syndrome (AIDS), acute disseminated encephalomyelitis (ADEM), Addison's disease, agammaglobulinemia, allergic diseases, alopecia areata, Alzheimer's disease, amyotrophic lateral sclerosis, ankylosing spondylitis, antiphospholipid syndrome, antisynthetase syndrome, arterial plaque disorder, asthma, atherosclerosis, atopic allergy, atopic dermatitis, autoimmune aplastic anemia, autoimmune cardiomyopathy, autoimmune enteropathy, autoimmune hemolytic anemia, autoimmune hepatitis, autoimmune hypothyroidism, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenia purpura, autoimmune urticarial, autoimmune uveitis, Balo disease/Balo concentric sclerosis, Behcet's disease, Berger's disease, Bickerstaffs encephalitis, Blau syndrome, bullous pemphigoid, Castleman's disease, celiac disease, Chagas disease, chronic inflammatory demyelinating polyneuropathy, chronic recurrent multifocal osteomyelitis, chronic obstructive pulmonary disease, chronic venous stasis ulcers, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's Syndrome, cutaneous leukocytoclastic angiitis, Dego's disease, Dercum's disease, dermatitis herpetiformis, dermatomyositis, Diabetes mellitus type I, Diabetes mellitus type II diffuse cutaneous systemic sclerosis, Dressler's syndrome, drug-induced lupus, discoid lupus erythematosus, eczema, emphysema, endometriosis, enthesitis-related arthritis, cosinophilic fasciitis, eosinophilic gastroenteritis, cosinophilic pneumonia, epidermolysis bullosa acquisita, erythema nodosum, erythroblastosis fetalis, essential mixed cryoglobulinemia, Evan's syndrome, fibrodysplasia ossificans progressive, fibrosing alveolitis (or idiopathic pulmonary fibrosis), gastritis, gastrointestinal pemphigoid, Gaucher's disease, glomerulonephritis, Goodpasture's syndrome, Graves' disease, Guillain-Barre syndrome (GBS), Hashimoto's encephalopathy, Hashimoto's thyroiditis, heart disease, Henoch-Schonlein purpura, herpes gestationis (aka gestational pemphigoid), hidradenitis suppurativa, HIV infection, Hughes-Stovin syndrome, hypogammaglobulinemia, infectious diseases (including bacterial infectious diseases), idiopathic inflammatory demyelinating diseases, idiopathic pulmonary fibrosis, idiopathic thrombocytopenia purpura, IgA nephropathy, inclusion body myositis, inflammatory arthritis, inflammatory bowel disease, inflammatory dementia, interstitial cystitis, interstitial pneumonitis, juvenile idiopathic arthritis (aka juvenile rheumatoid arthritis), Kawasaki's disease, Lambert-Eaton myasthenic syndrome, leukocytoclastic vasculitis, lichen planus, lichen sclerosus, linear IgA disease (LAD), lupoid hepatitis (aka autoimmune hepatitis), lupus erythematosus, lymphomatoid granulomatosis, Majeed syndrome, malignancies including cancers (e.g., sarcoma, Kaposi's sarcoma, lymphoma, leukemia, carcinoma and melanoma), Meniere's disease, microscopic polyangiitis, Miller-Fisher syndrome, mixed connective tissue disease, morphea, Mucha-Habermann disease (aka Pityriasis lichenoides et varioliformis acuta), multiple sclerosis, myasthenia gravis, myositis, narcolepsy, neuromyelitis optica (aka Devic's disease), neuromyotonia, occular cicatricial pemphigoid, opsoclonus myoclonus syndrome, Ord's thyroiditis, palindromic rheumatism, PANDAS (pediatric autoimmune neuropsychiatric disorders associated with streptococcus), paraneoplastic cerebellar degeneration, Parkinsonian disorders, paroxysmal nocturnal hemoglobinuria (PNH), Parry Romberg syndrome, Parsonage-Turner syndrome, pars planitis, pemphigus vulgaris, peripheral artery disease, pernicious anaemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restenosis, restless leg syndrome, retroperitoneal fibrosis, rheumatoid arthritis, rheumatic fever, sarcoidosis, schizophrenia, Schmidt syndrome, Schnitzler syndrome, scleroderma, sepsis, scleritis, scrum Sickness, Sjogren's syndrome, spondyloarthropathy, Still's disease (adult onset), stiff person syndrome, stroke, subacute bacterial endocarditis (SBE), Susac's syndrome, Sweet's syndrome, Sydenham chorea, sympathetic ophthalmia, systemic lupus erythematosus, Takayasu's arteritis, temporal arteritis (aka “giant cell arteritis”), thrombocytopenia, Tolosa-Hunt syndrome) transplant (e.g., heart/lung transplants) rejection reactions, transverse myelitis, tuberculosis, ulcerative colitis, undifferentiated connective tissue disease, undifferentiated spondyloarthropathy, urticarial vasculitis, vasculitis, vitiligo, and Wegener's granulomatosis.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, the term “synergistic effect” or grammatical variations thereof means and includes a cooperative action encountered in a combination of two or more active compounds in which the combined activity of the two or more active compounds exceeds the sum of the activity of each active compound alone. The term “synergistically effective amount,” as used herein, means and includes an amount of two or more active compounds that provides a synergistic effect defined above.

Disclosed herein are compositions and methods for promoting weight loss and/or preventing or obesity, and/or promoting health benefits in a subject. In certain aspects, the disclosed method comprises administering a composition to a subject where the composition comprises a postbiotic derived from Propionibacterium freudenreichii.

Propionibacterium freudenreichii is a gram-positive, non-motile bacterium that plays an important role in the creation of Emmental cheese, and to some extent, Jarlsberg cheese, Leerdammer and Maasdam cheese. Its concentration in Swiss-type cheeses is higher than in any other cheese. Propionibacterium strain, P. UF1, has been isolated from the gut microbiota of preterm infants. P. UF1 is also referred to herein as Propionibacterium freudenreichii UF1 or UF1.

In certain embodiments, the postbiotic derived from a bacterial strain is inactivated through a thermal process. Exemplary implementations of such thermal process include, but are not limited to pasteurization, sterilization, and/or ohmic heating. In further embodiments, the postbiotic derived from a bacterial strain is inactivated through a non-thermal process. Exemplary implementations, include, but are not limited to pulsed electric fields, ultrasound irradiation, and/or supercritical carbon dioxide.

In certain embodiments, at least one strain of bacteria is a probiotic.

In certain aspects, disclosed herein are methods to promote weight loss through the administration of an effective amount of one or more compositions disclosed herein.

According to certain aspects, administration of effective amounts of the disclosed compositions are used in treating diabetes mellitus; preventing, slowing progression of, delaying or treating of a condition or disorder selected from the group consisting of complications of diabetes mellitus; preventing, slowing the progression of, delaying or treating a metabolic disorder selected from the group consisting of type 1 diabetes mellitus, type 2 diabetes mellitus, impaired glucose tolerance (IGT), impaired fasting blood glucose (IFG), hyperglycemia, postprandial hyperglycemia, overweight, obesity, metabolic syndrome and gestational diabetes; or improving glycemic control and/or for reducing of fasting plasma glucose, of postprandial plasma glucose and/or of glycosylated hemoglobin HbA1c; or preventing, slowing, delaying or reversing progression from impaired glucose tolerance IGT), impaired fasting blood glucose (IFG), insulin resistance and/or from metabolic syndrome to type 2 diabetes mellitus; or preventing, slowing the progression of, delaying or treating of a condition or disorder selected from the group consisting of complications of diabetes mellitus such as cataracts and micro- and macrovascular diseases, such as nephropathy, retinopathy, neuropathy, tissue ischemia, arteriosclerosis, myocardial infarction, stroke and peripheral arterial occlusive disease; or reducing body weight and/or body fat or preventing an increase in body weight and/or body fat or facilitating a reduction in body weight and/or body fat; or preventing, slowing, delaying or treating diseases or conditions attributed to an abnormal accumulation of ectopic fat; or maintaining and/or improving the insulin sensitivity and/or for treating or preventing hyperinsulinemia and/or insulin resistance; preventing, slowing progression of, delaying, or treating new onset diabetes after transplantation (NODAT) and/or post-transplant metabolic syndrome (PTMS); preventing, delaying, or reducing NODAT and/or PTMS associated complications including micro- and macrovascular diseases and events, graft rejection, infection, and death; treating diabetes associated with cystic fibrosis treating hyperuricemia and hyperuricemia associated conditions; treating or prevention kidney stones; treating hyponatremia; in a patient in need thereof.

Another aspect encompasses a combination therapy to regulate fat storage, energy utilization, and/or weight loss in a subject. In an exemplary embodiment, a combination for increasing energy utilization, decreasing body fat or for promoting weight loss may include combining the methods and compositions disclosed with a procedure or therapy such as a pharmaceutical therapy, gastric bypass, duodenojejunal bypass, biliopancreatic diversion, vertical sleeve gastrectomy, adjustable gastric banding, vertical banded gastroplasty, intragastric balloon therapy, gastric plication, Magenstrasse and Mill, small bowel transposition, biliary diversion, brown adipose tissue modulation (e.g., controlled activation, enhanced differentiation, supplemental implantation, etc.), pharmaceutical administration, electrical stimulation of nerves that innervate at least a portion of the gastrointestinal tract, therapies impacting circadian rhythms, bile acid modulation, intestinal mucus production and metabolism, duodenal endoluminal barrier or similar manipulations of the gastrointestinal tract. For example, a composition comprising a postbiotic derived from UF1 can be administered to the subject prior to, concurrently with or after a gastric bypass or other gastrointestinal or bariatric procedure.

In certain aspects, administration of the disclosed compositions is effective at preventing reducing body weight and/or body fat or preventing an increase in body weight and/or body fat or facilitating a reduction in body weight and/or body fat; or preventing, slowing, delaying or treating diseases or conditions attributed to an abnormal accumulation of liver fat; or maintaining and/or improving the insulin sensitivity and/or for treating or preventing hyperinsulinemia and/or insulin resistance.

In certain embodiments, the composition is administered with a postbiotic dose of from about 1 mg to about 5,000 mg. In further embodiments, the postbiotic dose is from about 5 mg to about 4,000 mg. In yet further embodiments, the postbiotic dose is from about 25 mg to about 3,500 mg. In even further embodiments, the postbiotic dose is from about 50 mg to about 2,500 mg.

According to certain embodiments, fat loss is promoted through inducing thermogenesis in the subject. According to exemplary implementations of these embodiments, the composition may also include one or more compounds selected from: caffeine, green tea, capsaicin, garcinia cambogia, yohimbine and bitter orange.

According to still further embodiments, fat loss is promoted through enhancing lipolysis in the subject. In exemplary implementations of these embodiments, the composition further may include one or more compounds selected from caffeine, green tea extract, L-carnitine, Garcinia cambogia (hydroxycitric acid), capsaicin, ginseng, taurine, silk peptides and octacosanol.

According to certain implementations, the disclosed method further comprises restricting calorie intake of the subject. In exemplary implementations, the amount of fat loss in the subject is greater than that for a subject with an equivalent calorie restriction that has not been provided the composition. According to further implementations, the ratio of fat loss to muscle loss in the subject is greater than that for a subject with an equivalent calorie restriction that has not been provided the composition.

According to certain embodiments of the disclosed method, the composition is administered in a therapeutically effective amount. In further embodiments, the composition is administered in a prophylactically effective amount.

In some embodiments, contemplated methods may further comprise assessing one or more indices of on-going weight loss, e.g. the ketone body production level in a patient; and optionally adjusting the amount administered; thereby optimizing the therapeutic efficacy of the postbiotic derived from UF.

According to certain embodiments, administration of the disclosed compositions can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a weight loss target, is achieved. For example, when a loss of about 20% body weight, about 30% body weight or more has been achieved. A treatment regimen can include a corrective phase, during which a composition dose sufficient to provide reduction of excess adiposity is administered, followed by a maintenance phase, during which a lower postbiotic derived from UF1 dose sufficient to prevent re-development of excess adiposity is administered.

Additionally, administration of the disclosed composition promotes a health benefit in a subject by administering an effective amount of the composition. The health benefit can include promoting antimicrobial activity against gut pathogens, immune function, reducing inflammation, anti-allergenic function, anti-carcinogenic, mental health, memory cognition, intestinal epithelial barrier function, skin health, vaginal health, and/or oral health. The subject can be an infant, and the composition can be delivered as an infant formula, improving infant nutrition and supporting immune function.

Further disclosed herein is a method of inhibiting inflammation in a subject in need thereof comprising administering to the subject a composition a postbiotic derived from Propionibacterium freudenreichii. In certain embodiments, the subject is at risk of developing inflammatory disease or condition. In further embodiments, the subject has been diagnosed with an inflammatory disease or condition. In exemplary implementations, the subject has been diagnosed with diabetes, Crohn's disease, rheumatoid arthritis, fibromyalgia, systemic lupus erythematosus, glomerulonephritis, scleroderma, or multiple sclerosis.

In certain embodiments, administration of the disclosed postbiotic compositions reduces inflammatory cytokines in the subject. In certain embodiments, reduction of inflammatory cytokines occurs in the gut and/or intestinal tract of the subject. In certain implementations, interleukin-12 (IL-12) and/or tumor necrosis factor-alpha (TNF-α) are reduced in the subject. In exemplary implementations, administration of the composition to the subject produces a decrease in IL-12 and/or TNF-α levels in the subject relative to a control subject. In certain embodiments, IL-12 and/or TNF-α levels are decreased from about 20% to about 65%.

The disclosed compositions are formulated for oral administration including chewable foods, beverages, liquids, tablets, capsules, powders, and granulates. In a preferred embodiment the compositions have been formulated into a tablet. In another preferred embodiment the compositions have been formulated into a capsule. In yet another preferred embodiment the compositions have been formulated into granulated or water soluble powders. Further, in certain embodiments, compositions can be formulated into liquids, creams, lotions, gels dispersions or ointments for topical administration.

In certain embodiments, the disclosed postbiotics are administered in conjunction with a probiotic. When administered in conjunction with a probiotic, the composition provides a synergic effect relative to the administration of the postbiotic and the probiotic alone.

When formulated the composition may contain further ingredients, including ingredients that have a favorable impact on health, flavor, formulating or tableting. Non-limiting examples of additional ingredients that may suitably be incorporated in the present composition are: vitamins, minerals, nutritional supplements (e.g., fiber), fungal extracts, botanical extracts, sweeteners, flow aids, and fillers.

Tableting aids include for example, carboxylic acids such as malic, maleic, citric, iso-citric and succinic, and salts thereof, SiO₂, Aloe Vera, saturated and unsaturated linear and branched fatty acids and their salts, or fatty alcohols. Preferred tableting aides are malic acid, citric acid, stearic acid or Magnesium stearate. Tableting aides are formulated at a concentration of about 1% to 10%, w/w, 2.5% and 7.5 w/w % or any specific value within said range.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods.

As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

Compositions of the present disclosure may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

Various aspects and embodiments of the present disclosure are defined by the following numbered clauses:

1. A method for reducing body weight in a subject comprising administering to the subject a composition comprising: a postbiotic derived from at least one strain of bacteria wherein the at least one strain of bacteria comprises Propionibacterium freudenreichii UF (UF1).
2. The method of clause 1, wherein the composition is administered with a postbiotic dose of from about 1 mg to about 5,000 mg.
3. The method of clause 2, wherein the postbiotic dose is from about 5 mg to about 4,000 mg.
4. The method of clause 3, wherein the postbiotic dose is from about 25 mg to about 3,500 mg.
5. The method of clause 4, wherein the postbiotic dose is from about 50 mg to about 5,500 mg.
6. The method of clause 1, wherein the composition further comprises a probiotic.
7. The method of clause 1, wherein the composition further comprises a prebiotic.
8. The method of clause 1, wherein the at least one strain of bacteria comprises at least a second strain of bacteria chosen from: Propionibacterium acnes, Propionibacterium avidum, Propionibacterium granulosum, Propionibacterium lymphophilum, Propionibacterium acidiproprionic, Propionibacterium jensenii Propionibacterium thoenii, lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, and/or mixtures of the foregoing.
9. A method of managing a body weight disorder in a subject in need thereof comprising administering to the subject a therapeutically effective amount of a composition comprising: a postbiotic derived from at least one strain of bacteria wherein the at least one strain of bacteria comprises Propionibacterium freudenreichii UF (UF1).
10. The method of clause 9, wherein the composition is administered with a postbiotic dose of from about 1 mg to about 5,000 mg.
11. The method of clause 10, wherein the postbiotic dose is from about 5 mg to about 4,000 mg.
12. The method of clause 11, wherein the postbiotic dose is from about 25 mg to about 3,500 mg.
13. The method of clause 12, wherein the postbiotic dose is from about 50 mg to about 5,500 mg.
14. The method of clause 9, wherein the composition further comprises a probiotic.
15. The method of clause 9, wherein the body weight disorder is one or more of obesity, lypodystrophy, diabetes or metabolic syndrome, and fibrosis.
16. The method of clause 15, wherein the composition is administered in conjunction with calorie restriction of the subject.
17. The method of clause 16, wherein the combined administration of the composition and calorie restriction results in greater weight loss in the subject than the weight loss produced by calorie restriction alone.
18. A method promoting a health benefit in a subject comprising administering to the subject an effective amount of a composition comprising: a postbiotic derived from at least one strain of bacteria wherein the at least one strain of bacteria comprises Propionibacterium freudenreichii UF (UF1).
19. The method of clause 18, wherein the composition is administered with a postbiotic dose of from about 1 mg to about 5,000 mg.
20. The method of clause 19, wherein the postbiotic dose is from about 5 mg to about 4,000 mg.
21. The method of clause 20, wherein the postbiotic dose is from about 25 mg to about 3,500 mg.
22. The method of clause 21, wherein the postbiotic dose is from about 50 mg to about 5,500 mg.
23. The method of clause 18, wherein the composition further comprises a prebiotic.
24. The method of any of clauses 18-23, wherein the health benefit is promoting one or more of: antimicrobial activity against gut pathogens, immune function, reduces inflammation, anti-allergenic function, anti-carcinogenic, mental health, memory, cognition, intestinal epithelial barrier function, skin health, vaginal health, and/or oral health.
25. The method of clause 24, wherein the subject is an infant.
26. The method of clause 25, wherein the composition is delivered as an infant formula.
27. The method of clause 25, wherein the composition improves infant nutrition.
28. The method of clause 25, wherein the one or more health benefit is supporting immune function.
29. The method of clause 28, wherein the more or more health benefit is promoting immune function in an immunocompromised subject.
30. A method of inhibiting inflammation in a subject in need thereof comprising administering to the subject a composition comprising a postbiotic derived from at least one strain of bacteria wherein the at least one strain of bacteria comprises Propionibacterium freudenreichii UF (UF1).
31. The method of clause 30, wherein the composition is administered with a postbiotic dose of from about 1 mg to about 5,500 mg.
32. The method of clause 31, wherein the composition is administered with a postbiotic dose of from about 50 mg to about 3,500 mg.
33. The method of clause 32, wherein the composition further comprises a prebiotic.
34. The method of clause 30, wherein the subject has been diagnosed with an inflammatory disease or condition.
35. The method of clause 34, wherein the subject has been diagnosed with diabetes, Crohn's disease, rheumatoid arthritis, fibromyalgia, systemic lupus erythematosus, glomerulonephritis, scleroderma, or multiple sclerosis.
36. The method of any preceding clause, wherein the at least one strain of bacteria comprises at least a second strain of bacteria chosen from: Propionibacterium acnes; Propionibacterium avidum; Propionibacterium granulosum; Propionibacterium lymphophilum; Propionibacterium acidiproprionic; Propionibacterium jensenii Propionibacterium thoenii; lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, and/or mixtures of the foregoing.
37. The method of any preceding clause, wherein the composition comprises a postbiotic and the postbiotic is derived from a bacterial strain inactivated through a thermal process.
38. The method of clause 37, wherein the thermal process comprises one or more of pasteurization, sterilization, and/or ohmic heating.
39. The method of any preceding clause, wherein the composition comprises a postbiotic and the postbiotic is derived from a bacterial strain inactivated through a non-thermal process.
40. The method of clause 39, wherein the non-thermal process comprises one or more of pulsed electric fields, ultrasound, irradiation, and/or supercritical carbon dioxide.
41. The method of any preceding clause, wherein the composition is administered in conjunction with a probiotic and provides a synergic effect relative to the administration of the postbiotic and the probiotic alone.

### EXAMPLES

The following example(s) are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

### Experiment Design

Nine mice (n=9) were orally gavaged with phosphate-buffered saline (PBS) (n=5) or heat-killed Propionibacterium freudenreichii P.UF1 (HK-P.UF1) (10\hat{ }9 CFU per dose, n=4) for two weeks. Colonic dendritic cells (DCs) were analyzed using flow cytometry (fluorescence-activated cell sorting, FACS), which sorts cells based on their light scattering and fluorescent properties.

### RESULTS

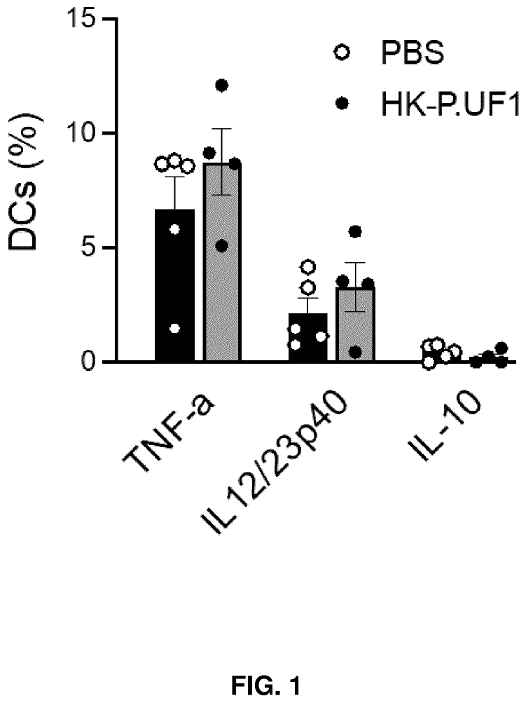

As shown in FIG. 1, ingestion of HK-P.UF1 led to unexpectedly beneficial changes in the cytokine profile produced by colonic DCs. Compared to the PBS control, HK-P.UF1 reduced the inflammatory cytokine interleukin-12 (IL-12) and tumor necrosis factor-alpha (TNF-α), while increasing the anti-inflammatory cytokine interleukin-10 (IL-10).

Although the disclosure has been described with reference to preferred embodiments, persons skilled in the art will recognize that changes may be made in form and detail without departing from the spirit and scope of the disclosed apparatus, systems and methods.

What is claimed is:

1. A method for reducing body weight in a subject comprising administering to the subject a composition comprising: a postbiotic derived from at least one strain of bacteria wherein the at least one strain of bacteria comprises Propionibacterium freudenreichii UF (UF1).

2. The method of claim 1, wherein the composition is administered with a postbiotic dose of from about 1 mg to about 5,000 mg.

3. The method of claim 2, wherein the postbiotic dose is from about 5 mg to about 4,000 mg.

4. The method of claim 3, wherein the postbiotic dose is from about 25 mg to about 3,500 mg.

5. The method of claim 4, wherein the postbiotic dose is from about 50 mg to about 2,500 mg.

6. The method of claim 1, wherein the composition further comprises a probiotic.

7. The method of claim 1, wherein the composition further comprises a prebiotic.

8. The method of claim 1, wherein the at least one strain of bacteria comprises at least a second strain of bacteria chosen from: Propionibacterium acnes, Propionibacterium avidum, Propionibacterium granulosum, Propionibacterium lymphophilum, Propionibacterium acidiproprionic, Propionibacterium jensenii Propionibacterium thoenii, lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, and/or mixtures of the foregoing.

9. A method of managing a body weight disorder in a subject in need thereof comprising administering to the subject a therapeutically effective amount of a composition comprising: a postbiotic derived from at least one strain of bacteria wherein the at least one strain of bacteria comprises Propionibacterium freudenreichii UF (UF1).

10. The method of claim 9, wherein the composition is administered with a postbiotic dose of from about 1 mg to about 5,000 mg.

11. The method of claim 9, wherein the composition further comprises a probiotic.

12. The method of claim 9, wherein the body weight disorder is one or more of obesity, lypodystrophy, diabetes or metabolic syndrome, and fibrosis.

13. The method of claim 12, wherein the composition is administered in conjunction with calorie restriction of the subject and wherein the combined administration of the composition and calorie restriction results in greater weight loss in the subject than the weight loss produced by calorie restriction alone.

14. A method of inhibiting inflammation in a subject in need thereof comprising administering to the subject a composition comprising a postbiotic derived from at least one strain of bacteria wherein the at least one strain of bacteria comprises Propionibacterium freudenreichii UF (UF1).

15. The method of claim 14, wherein the composition is administered with a postbiotic dose of from about 1 mg to about 5,500 mg.

16. The method of claim 15, wherein the composition is administered with a postbiotic dose of from about 50 mg to about 3,500 mg.

17. The method of claim 16, wherein the composition further comprises a prebiotic.

18. The method of claim 17, wherein the subject has been diagnosed with an inflammatory disease or condition.

19. The method of claim 18, wherein the subject has been diagnosed with diabetes, Crohn's disease, rheumatoid arthritis, fibromyalgia, systemic lupus erythematosus, glomerulonephritis, scleroderma, or multiple sclerosis.

20. The method of claim 14, wherein administration of the composition promotes one or more further health benefit to the subject selected from: enhanced antimicrobial activity against gut pathogens, enhanced immune function, anti-allergenic function, anti-carcinogenic, enhanced mental health, enhanced memory, enhanced cognition, enhanced intestinal epithelial barrier function, enhanced skin health, enhanced vaginal health, and/or oral health.
